(19) United States
(12) Patent Application Publication
(10) Pub. No.: US 2006/0115818 A1
(43) Pub. Date: Jun. 1, 2006

(54) Diagnostic and therapeutic means for kidney stone related pathologies

(76) Inventors: Mario Pirastu, Cagliari (IT); Fernando Gianfrancesco, Cagliari (IT); Teresa Esposito, Cagliari (IT); Maria Neve Ombra, Cagliari (IT)

Correspondence Address: Albert Wai Kit Chan;Law Offices of Albert Wai Kit Chan, World Plaza Suite 604, 141 07 20th Avenue, Whitestone, NY, 11357, US

(21) Appl. No.: 10/531,637

(22) PCT Filed: Oct. 14, 2003
(86) PCT No.: PCT/IT03/00627

§ 371 Date: Sep. 12, 2005

(30) Foreign Application Priority Data
Oct. 15, 2002 (IT) RM2002A000525

(51) Int. Cl. C12Q 1/68; C12P 19/34; C07H 21/04

(52) U.S. Cl. 435/6; 435/91.2; 536/23.1

(57) ABSTRACT

A description is given of a nucleic acid comprising at least one fragment of the human gene ZNF365 or functional portions thereof for use in the diagnosis or treatment of pathologies associated with kidney stones. Appropriate diagnostic kits are also described.

The present invention concerns diagnostic and therapeutic means for pathologies associated with kidney stones.

Nephrolithiasis is a common multifactorial disorder characterized by the presence of calculi (stones) in the urinary tract; an important genetic contribution to its pathogenesis has been established; its aetiology is unknown (Jaeger 1996; Curhan et al. 1997; Baggio 1999; Scheinman 1999). Kidney stones are estimated to affect 10% of the population (Serio and Fraioli 1999; Rivers et al. 2000). The major classes of stones are calcium oxalate, calcium phosphate, uric acid, struvite, and cystine. Uric acid nephrolithiasis (UAN) accounts for 20% of all stones. Formation of calculi may occur when the urine becomes overly concentrated with uric acid that may complex to form small crystals and subsequently stones. However, the molecular basis for urate handling in the human kidney is not completely clear.

The authors of the invention have previously identified in patients in the small Sardinian village of Talana a locus of approximately 2.5 cM potentially correlated with UAN and located on chromosome 10q21-q22 (Ombra et al. 2001). However, the locus cannot be used for screening assays on populations, nor for the development of diagnostic assays, nor to define therapeutic agents. It is therefore vital to identify the genomic region associated with the pathology.

To identify the region, the authors have extended the search to a larger sample of affected individuals selected from the same study group. Using mapping methods based on sharing of alleles and/or haplotypes identical by descent or on autozygosity, a linkage disequilibrium (LD) block of about 200 kb was found in the Talana population and a core region of 67 kb already associated with UAN. The authors have identified in the associated region a novel gene, called ZNF365, divided into 15 exons spanning a genomic region of about 300 kb and encoding for at least four different protein isoforms of 407, 333, 462 and 216 amino acids. The encoding sequence for the protein isoform of 216 amino acids was found to reside completely within the core interval of 67 kb. The protein has at least one transmembrane domain and various sites for N-terminal N- and O-glycosylation, suggesting that it may be a membrane protein. Mutation analysis showed that a mutation of a coding nucleotide, which causes a missense in exon 12 (Ala62Thr), is strongly associated with UAN (p=0.0096). Moreover, the variant protein has a different secondary structure and might be involved in the aetiology of UAN.

Hence, the object of the present invention is a nucleic acid comprising at least one fragment of the human gene ZNF365, in which the fragment encodes for a functional fragment of at least one of the proteins of the ZNF365 group to be used in the diagnosis of pathologies associated with kidney stones.

A further object of the invention is a nucleic acid comprising at least a fragment of the human gene ZNF365, in which the fragment encodes for a functional portion of at least one of the proteins of the ZNF365 group for therapeutic use in pathologies associated with kidney stones.

A further object of the invention is a method to detect in an individual at least one mutation of the gene encoding for one of the proteins of the ZNF365 group located on chromosome 10, comprising the phases:

collection of a sample containing a sufficient quantity of DNA from the aforesaid individual or reproducible in culture;

isolation of the DNA of the collected sample;

submission of the isolated DNA to exponential amplification using as an primer pair for the amplification reaction at least two oligonucleotides that are able to amplify at least one fragment of the human gene ZNF365, in which the fragment encodes for a functional portion of at least one of the proteins of the ZNF365 group;

detection of any mutations in at least one amplified fragment compared with healthy controls.

Preferably, the DNA exponential amplification phase is performed using primer pairs able to amplify a part of the fragment encoding the human gene ZNF365. More preferably, the DNA exponential amplification phase to amplify a part of the fragment encoding the human gene ZNF365 comprises the use of the following primer pairs:

| | |
|---|---|
| Ala62Thr-F: | 5′ CTC CAC TCC ACC TTT TTA AG 3′ |
| Ala62Thr-R: | 5′ GCT GAC ATT GGT ACT TAC TG 3′. |

More preferably, the detection phase of any mutations in at least one amplified fragment compared with healthy controls is performed using direct sequencing. An expert from the field will know which alternative methods come under the range of protection of the invention.

A further object of the invention is a diagnostic kit for pathologies associated with kidney stones and comprises:

at least one pair of oligonucleotide primers for the exponential amplification reaction of at least one fragment of the human gene ZNF365, in which a fragment encodes for a functional portion of at least one of the proteins of the ZNF 365 group;

a control DNA from a healthy individual not affected by pathologies associated with kidney stones.

Preferably, the oligonucleotide primer pairs in the kit for performing the amplification reaction are able to amplify a part of the region encoding the ZNF365 gene.

A further object of the invention is a protein belonging to the group of ZNF365 proteins or a functional portion thereof for use in the diagnosis of pathologies associated with kidney stones.

A further object of the invention is a protein belonging to the group of ZNF365 proteins or a functional portion thereof for use in the treatment of pathologies associated with kidney stones.

The invention is described below with broad examples and in reference to the following figures:

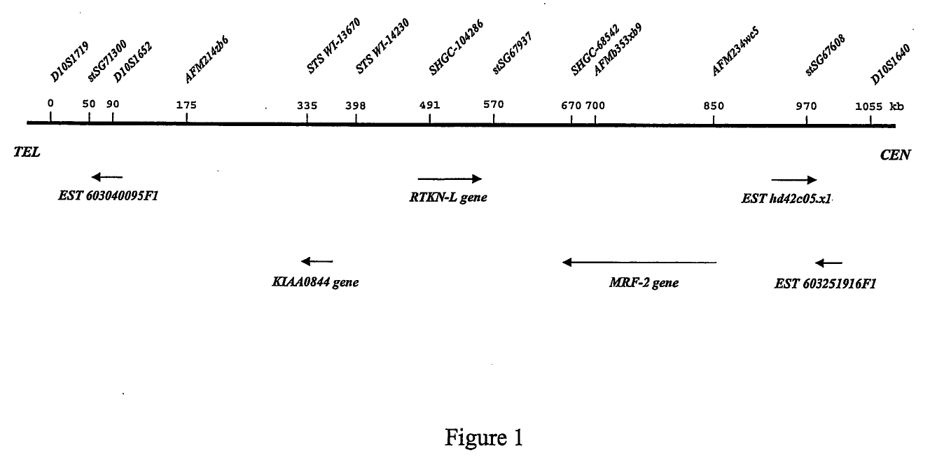

FIG. 1. Physical and transcriptional map of the UAN critical region. Markers of the physical map are indicated vertically above the solid bar representing genomic DNA. Microsatellites used in linkage analysis are D10S1719, D10S1652 and D10S1640. All genes and ESTs located in this region are shown.

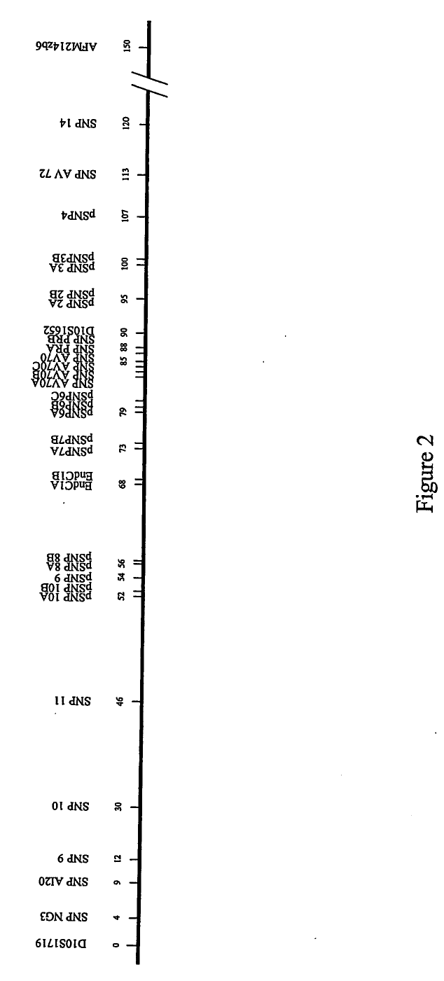

FIG. 2. SNPs map of the LD block.

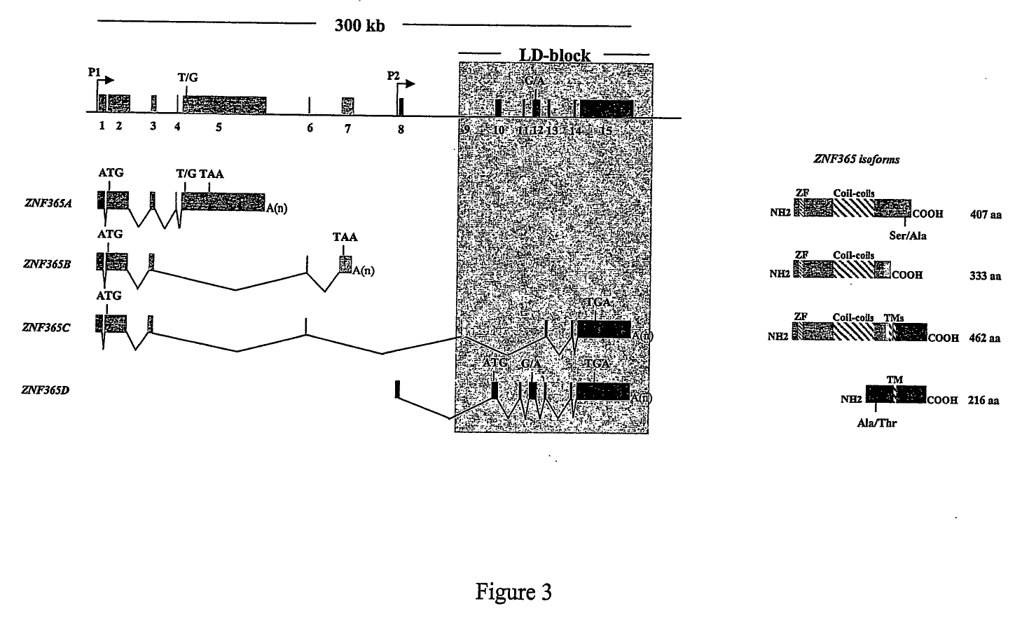

FIG. 3. Genomic structure of the ZNF365 gene showing alternative splicing and alternative start sites that generate four different transcripts. Initiation codons (ATG) and stop codons (TM or TGA) are shown for all isoforms. Two different promoters, a CpG island (P1) and a TATA box (P2) are indicated with arrows. Exons used for each transcript are indicated as colored boxes, while the LD block is indicated as a grey box. Two missense variants found in exons 5 and 12 are shown.

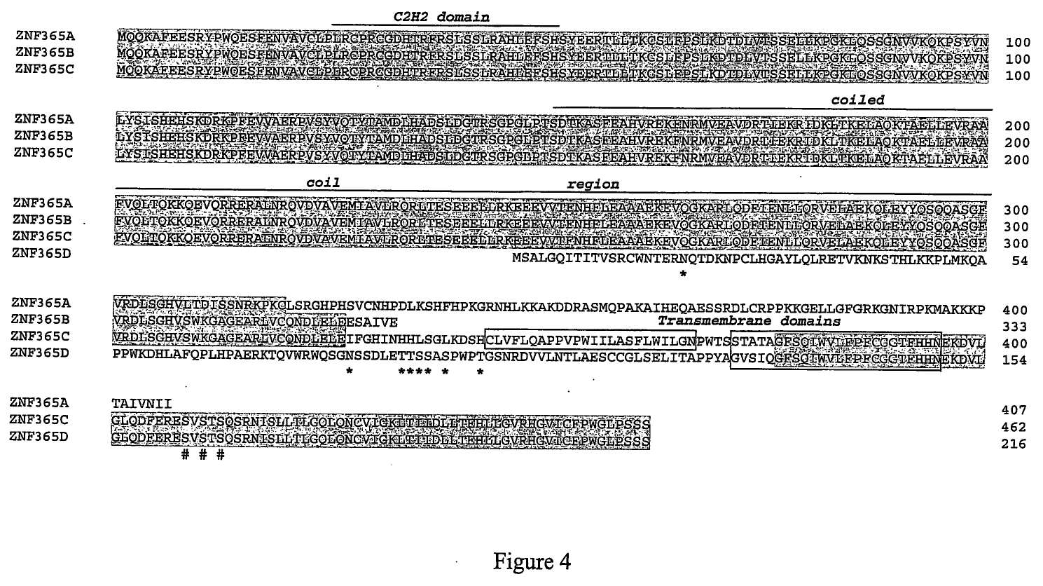

FIG. 4. Relationship between the amino acid sequences of ZNF365 proteins. Grey boxes denote identical amino acids. Solid lines indicate the C2H2 zinc finger domain and coiled-coil fragments. The predicted transmembrane domains are shown in the rectangle.

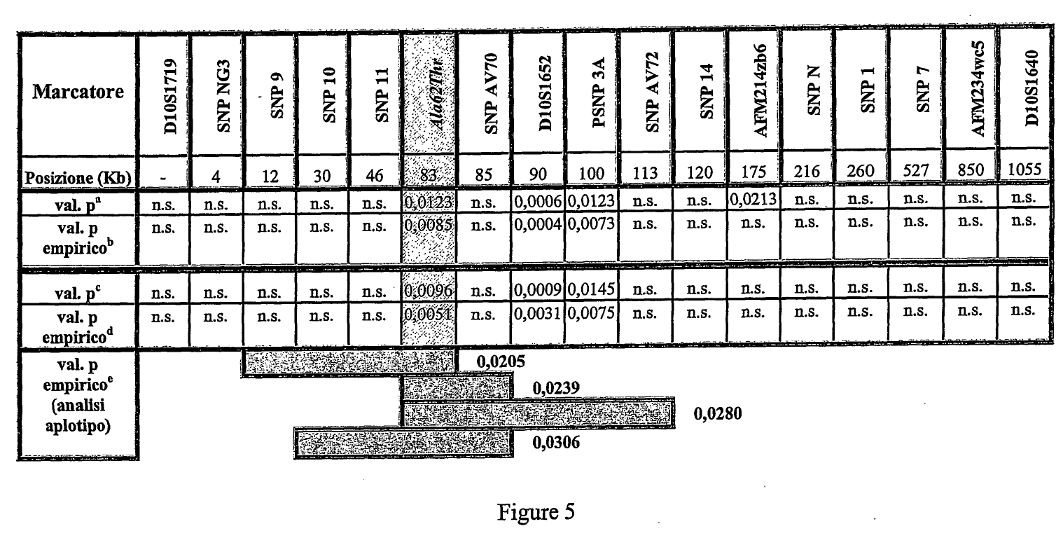

FIG. 5. Results of the association study. The molecular markers used in the study are indicated at the top. The position on the physical map and the association results are indicated for each marker. The different configurations of the associated haplotypes are shown at the bottom.

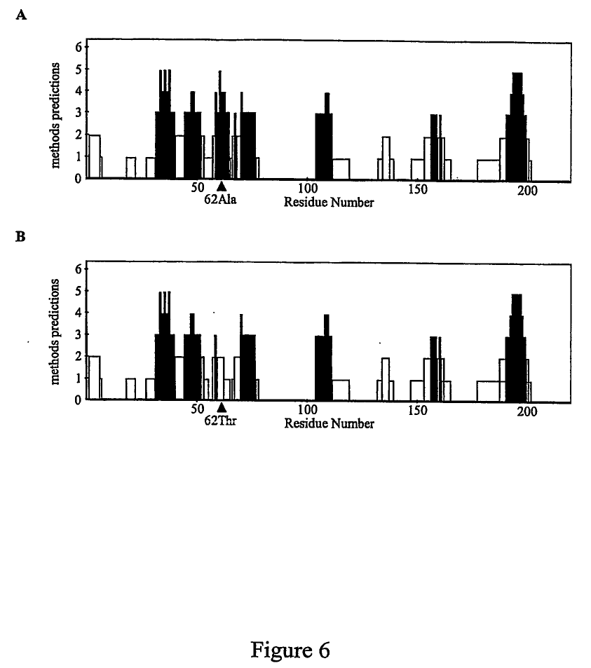

FIG. 6. Secondary structure predictions using the SecStr package are shown. The most probable structures, predicted by three or more methods, are colored black in the joint prediction histograms. (A) The structural prediction for the non associated allele (ala 62). (B) The structural prediction for the uric acid nephrolithiasis susceptibility allele (thr62).

### SUBJECTS AND METHODS

Sample Collection

All individuals participating in the study came from an isolated village (Talana) in central Sardinia (1200 inhabitants) that is characterized by slow population growth, high endogamy, and high inbreeding (Wright et al. 1999; Angius et al. 2001). The diagnosis of UAN was clinically confirmed by accurate physical examination and by renal ultrasonography. The original patient list included 134 affected subjects. For this study we selected 62 patients (mean age 58.4 years) with severe UAN. All subjects gave signed informed consent, and all samples were taken according to the Helsinki Declaration.

SNP Genotyping

To find common SNPs in the UAN critical region, we performed an extensive alignment of genomic sequences deposited in public databases using Sequencher software. The screening panel included 10 unrelated persons from the Talana population. PCR amplicons of 800-1000 nt containing the selected putative polymorphisms were designed using the Oligo 4.0 software program. PCR reactions were prepared using 50 ng of genomic DNA template, 0.5 μM of each PCR-primer, 1.5 mM MgCl₂, 0.2 mM of each dNTP, 0.5 U Taq polymerase and the buffer recommended by the supplier (Amersham) in a final volume of 25 μl. Thermo cycling started with a single denaturation step for 2 min at 94° C., followed by 35 cycles of denaturation for 30 sec at 94° C., annealing for 30 sec at Temperature Melting (TM) and extension at 72° C. for 45 sec. One final extension step was added for 7 min at 72° C.

Samples were then sequenced using the Big Dye Terminator Ready Reaction Kit (Applied Biosystem). Sequencing reactions were performed on a 9700 Thermal Cycler (Applied Biosystems) for 25 cycles at 95° C. for 10 sec, TM for 5 sec and at 60° C. for 2 min. After the sequencing, each reaction was isopropanol-precipitated. Sequencing of the products was performed on the ABI prism 3100 Genetic Analyser (Applied Biosystems). Polymorphisms were detected by multiple alignments of sequences using the Autoassembler program (Applied Biosystems).

Mutational analysis of all 15 exons of the identified ZNF365 gene was performed using PCR, including the intron/exon boundaries.

Genotyping of the single nucleotide polymorphisms was performed by dot-blot hybridisation of the PCR products, comprising the polymorphisms, with short allele-specific oligonucleotides (ASO probes) (Ristaldi et al. 1989).

cDNA Clones

ZNF365A (KIAA0844 accession number ABO20651; http://www.kazusa.or.jp/huge/) was provided by the Kazusa DNA Research Institute. ZNF365B cDNA sequence was obtained by sequencing the cDNA clones 3069791 and 4821260 from the IMAGE Consortium. ZNF365C and ZNF365D cDNA sequences were obtained by RT-PCR/TOPO-cloning vector (Invitrogen) strategy using primer pairs in different exons of the gene on human kidney RNA (Clontech) and 5′- and 3′-RACE strategy carried out using a human kidney Marathon-Ready cDNA kit.

RNA Expression Studies

A human multiple-tissue Northern blot (Clontech) was hybridized with ZNF365A, B, C and D transcripts. The Northern blot was prehybridized, hybridized, and washed according to the manufacturers directions (Clontech).

RT-PCR analysis: 0.5 μg of human kidney (Clontech), adrenal gland (Clontech) and blood mRNA were reverse-transcribed in a 50 μl reaction mixture containing 1× RT buffer (20 mM Tris-HCl pH 8.4, 50 mM KCl, 2.5 mM MgCl₂), 10 mM DTT, 0.5 mM dNTP, 0.2 μg of random hexamers (Roche), and 200 units of SuperScript reverse transcriptase (Invitrogen). After 60 min incubation at 37° C., 1 μg of DNase-free RNase was added and incubated 10 min at 37° C. The cDNA was extracted twice with phenol/chloroform/iso-amylalcohol (25:24:1). The cDNA was then precipitated overnight with 1/10 vol of 3 M sodium acetate, pH 5.2, and 2.5 vol of ethanol. 10 ng of each cDNA were used as templates in a 25 μl PCR reaction containing 1× PCR buffer (Amersham), 0.2 mM dNTPs, 0.5 units of AmpliTaq polymerase (Amersham), and 0.5 μM each of the primer sequences derived from the cDNA. Using a DNA Thermal Cycler 9700 (Applied Biosystems), we carried out 40 amplification cycles for 30 sec at 94° C., 30 sec at TM, and 45 sec at 72° C.

5′ and 3′ RACE strategy was carried out by using 5 μl of Marathon human kidney cDNA (Clontech),1× PCR buffer (Amersham), 0.2 mM dNTPs, 1.0 units of AmpliTaq polymerase (Amersham), and 0.5 μM each of the gene specific primer and AP1 primer (Clontech) in a final volume of 50 μl. 25 amplification cycles were performed for 30 sec at 94° C., 20 sec at TM, and 4 min at 68° C. Nested PCRs were carried out with the same protocol described for the first PCRs using 1/250 of the first PCRs, gene specific primers 2 and AP2 primer (Clontech) in a final volume of 50 μl. 30 amplification cycles were carried out for 30 sec at 94° C., 20 sec at TM, and 4 min at 68° C. Aliquots of the first and second PCR reactions performed with different gene specific primers were run on agarose gel and blotted on nylon membrane according to the manufacturer's directions (Amersham). The membranes were hybridized with a PCR product including exons 2, 3, 4 and 5 of the ZNF365D transcript. Positive PCR products were cloned in TOPO-vector (Invitrogen) and sequenced with M13 forward and reverse primers.

Results

Transcriptional Map of UAN Locus

We obtained a consensus genomic sequence of the 1.1 Mb region, corresponding to the identified 2.5 cM critical interval, by alignment of partial sequences deposited in different databases. The 1.1 Mb interval was found to contain at least 6 novel genes including 2 uncharacterised ones. (FIG. 1). MRF-2 (modulator recognition factor 2) gene is encoded by 10 exons and spans a region of about 200 kb. This gene may be an ortholog of mouse Desrt. Homozygous mutants of this gene are developmentally and sexually retarded and have transient immune abnormalities (Lahoud et al. 2001). RTKN-L (rhotekin like) gene is a homologue of RTKN gene, an inhibitor of rho GTPase activity, located on chromosome 2 (Fu et al. 2000). RTKN-L consists of at least 10 exons and spans a region of 75 kb. Given their biological function, these two genes were not obvious candidates for uric acid nephrolithiasis. The EST 603251916F1 sequence (GenBank Acc. BI603606) belongs to the UniGene cluster Hs.252954. By using the longest mRNA sequence of this UniGene cluster, we found that this gene is split in at least five exons and spans a region of 50 kb. The EST hd42c05.×1 (GenBank Acc. AW511012) exhibits no significant similarity to any ESTs deposited in public databases, suggesting that the gene is present at very low levels in human cells. KIAA0844 cDNA is derived from the HUGE protein database (Nagase et al. 2000). Finally, the EST 603040095F1 (GenBank Acc. BI822044) encodes for five exons and spans a small region of 15 kb. In the absence of a functional assignment, it is not possible to know which one of these 4 orphan genes may play a role in the pathogenesis of UAN, even though the last EST is the closest to marker D10S1652 already found to be associated with UAN.

Molecular Characterization of the UAN Susceptibility Gene

All previously described genes except the EST 603040095F1 were located outside the LD block and so none of them were further analysed. In order to characterize the 5′ and 3′ regions of this gene, we performed RT-PCR and RACE strategies starting from the coding region of the EST 603040095F1. An extensive gene spanning a 300 kb region and overlapping the LD block was disclosed. Genomic and cDNAs sequence comparisons showed that the gene consists of 15 exons and includes the EST 603040095F1 inside the critical region (delimited by AFM214zb6) and KIAA0844 outside it (FIG. 1). In the human kidney, where we worked on a complex pattern of alternative splicing and transcriptional start sites, different proteins of 407, 333, 462 and 216 amino acids are generated (FIG. 3, Table 1).

TABLE 1

| | | | | |
|---|---|---|---|---|
| Exon | (bp) | Exon-Intron junctions | Intron | (bp) |

| | | | | |
|---|---|---|---|---|
| 1 | 264 | gagcaagggtGTTCCCCGCG . . . AACAAGTCGGgtaagaggcg | 1 | 1721 |
| 2 | 756 | cccctcccagGACTTTTAGA . . . GGAAAGAAGAgtaagtgttg | 2 | 11459 |
| 3 | 181 | tgccttgcagAGAAGTTGTC . . . CGGGCACGTGgtgagtcacc | 3 | 10160 |
| 4 | 38 | tttccttcagCTTACAGACA . . . CCAAATGCCTgtatgtatgt | 4 | 750 |
| 5 | 2920 | ccctctgtagAAGCCGAGGG . . . AGCTGTTAAGaattcctgct | 5 | 57322 |
| 6 | 57 | ttcctttcagAGCTGGAAAG . . . GGAATTGGAGgtaaagccac | 6 | 20079 |
| 7 | 413 | ttatgactagGAGTCTGCGA . . . TGAGTAATTTatttattaag | 7 | 40174 |
| 8 | 150 | agctttgtaaGAAGAGGAAG . . . CTCCATAACCgtaagaaata | 8 | 102482 |
| 9 | 148 | ttgtttttagATTTTTGGCC . . . GCTACTGCAGgtgaatactc | 9 | 20497 |
| 10 | 222 | tattttacagGAATTGGCCC . . . CTGTCTCCAGgtaaattccc | 10 | 10880 |
| 11 | 88 | attattctagGTGCTGGAAT . . . CAAAAACAAGgtaagtcagt | 11 | 436 |
| 12 | 265 | ttccacaaagTCAACACATC . . . TCAATTCAAGgtcatttttg | 12 | 764 |
| 13 | 91 | tctgtttcagGATTTAGCCA . . . ACTTTGAGAGgtgggtgtcc | 13 | 9642 |
| 14 | 74 | atctctgcagAGAAAGTGTC . . . TGTGTGATTGgtaagaattt | 14 | 4006 |
| 15 | 1805 | gtcttcatagGCAAATTGAC . . . TACACAGTGTatacagtttt | | |

ZNF365A transcript—KIAA0844 cDNA (4158 bp) is encoded by 5 exons and its putative promoter region contains a CpG island. The open reading frame (ORF) of 1224 bp encodes a putative protein of 407 amino acids with a molecular weight of 46558 Da. The N-terminal region of this protein (amino acids 26-51) harbours the classical zinc finger domain of C2H2 family (FIG. 4). This domain is found in numerous nucleic acid-binding proteins and has also been used in protein-protein interactions and membrane associations (Laity et al. 2001). This gene was renamed ZNF365A (zinc finger protein 365) by the HUGO Gene Nomenclature Committee. A secondary structure prediction for this protein suggests the presence of four predicted alpha-helical coiled-coil domains frequently used as as oligomerization motif.

ZNF365B transcript—This transcript of 1671 bp, was identified by EST analysis (associated ESTs are 602688686F1 and UI-H-BW1-amf-e-12-0-UI.s1) and, like ZNF365A, was located outside the critical region. Unlike ZNF365A, this transcript utilizes different exons 4 and 5 (FIG. 3). The encoded protein of 333 amino acids is identical to the ZNF365A protein in the N-terminal region (exons 1-3) containing the zinc finger domain and coiled-coil fragments but it is different at the C-terminal (FIG. 4).

ZNF365C transcript—ZNF365C was isolated by RT-PCR analysis using different combinations of primers anchored in different exons of the gene. This transcript of 3376 bp overlaps the critical region. It uses the first 4 exons and the same CpG island promoter of ZNF365B transcript outside the critical region and 4 different exons (9, 13, 14 and 15) located in the LD block (FIG. 3). The encoded protein of 462 amino acids is similar to the ZNF365B protein but has a different C-terminal region. Computer-assisted analysis of this isoform based on transmembrane domain prediction revealed at least two membrane-spanning domains in this novel C-terminal region from amino acid 344 to 395, suggesting that it may be an integral membrane protein (FIG. 4).

ZNF365D transcript—ZNF365D spans a region of about 150 kb and uses its own specific TATA-box promoter. All coding exons of this transcript are completely included in the LD block associated with UAN (FIG. 3). Full-length cDNA was obtained by 5′ and 3′ RACE strategies. The composite cDNA sequence of 2695 bp encodes a protein of 216 amino acids that is completely different from the proteins encoded by ZNF365A and B, but identical to the ZNF365C protein in the C-terminal region (FIG. 4). Prediction analysis revealed a strong transmembrane domain in this isoform at positions 126-149, suggesting that, like ZNF365C, it could be an integral membrane protein with N-termini outside the cell. In this non cytosolic portion several potential modification sites were detected, including possible N and O-linked glycosylation sites (at Asn-20, Asn-82, Thr-88, Thr-89, Ser-90, Ser-91, Ser-93, Thr-97), suggesting that this outer-membrane protein portion may be glycosylated. In addition, several cysteine residues that may participate in disulfide bond formation were disclosed at positions 14, 27, 112, 113, 142, 183; and 207. Finally, three possible protein kinase C sites at Ser 163, Ser-165, Ser-167 at C-termini were shown (FIG. 4). These latter consensus sites may be located intracellularly.

The protein isoforms of 407, 333, 462 and 216 amino acids revealed no significant homology to other proteins deposited in public databases, suggesting that they could be a new class of undisclosed proteins. ZNF365B, C, D, revealed no signals by northern analysis, but RT-PCR assays showed low levels of expression in the blood, the kidney and the adrenal gland tissues. In particular, ZNF365D transcript was expressed in blood tissues 10 times less than GAPDH gene expression in a real-time PCR experiment.

A Specific ZNF365D Allele with an Altered Structure

Sequencing of all exons of ZNF365 and part of the intronic regions was performed on eight selected patients carrying the putative high-risk nephrolithiasis susceptibility haplotype. Two variants causing missense in the exon 5 (ZNF365A transcript) were identified that lead to serine/alanine amino acid substitution (variant Ser337Ala) and in the exon 12 (ZNF365D transcript) that lead to alanine/threonine amino acid substitution (variant Ala62Thr) (FIG. 3). Moreover, the authors identified a number of polymorphisms in the UTR regions and in intronic regions of the gene.

An analysis was performed to determine whether the presence of these alleles confers increased risk to UAN. A comparison of allelic frequencies in UAN patients and controls showed a strong association between UAN and variant Ala62Thr (p=0.0096; empirical p=0.0052). When haplotype frequencies in cases and controls including the identified variants were compared, we observed increased evidence of association with UAN for the various haplotype configurations (p=0.0205; p=0.0239; p=0.0280; p=0.0306) (FIG. 5).

Computer-assisted analysis strongly predicted that the ala62thr variation induces secondary structural alteration of the ZNF365D protein in the third alpha helical loop (FIG. 6). These results suggest that the effect of threonine causes a significant conformational change that may have important implications for the biological function of this protein or its interaction with other proteins.

### REFERENCES

Angius A, et al. (2001) Archival, demographic and genetic studies define a Sardinian sub-isolate as a suitable model for mapping complex traits. Hum Genet 109:198-209.

Baggio B (1999) Genetic and dietary factors in idiopathic calcium nephrolithiasis: what do we have, what do we need? J Nephrol 12:371-374.

Berger B, et al (1995) Predicting Coiled Coils by Use of Pairwise Residue Correlations. Proc Natl Acad Sci USA 92:8259-8263.

Bourgain C, et al (2000) Search for multifactorial genes in founder populations. Ann Hum Genet 64: 255-265.

Burgess A W, Ponnuswamy P K, Scheraga H A (1974) Analysis of conformations of aminoacid residues and prediction of backbone topography in proteins. Isr J Chem 12: 239-286.

Chou P. Fasman G D (1978) Prediction of the secondary structure of proteins from their aminoacid sequence. Adv Enzymol 47:45-148.

Cserzo M, et al (1997) Prediction of transmembrane alpha-helices in prokaryotic membrane proteins: the dense alignment surface method. Protein Eng 10: 673-676.

Curhan G C, et al (1997) Family history and risk of kidney stones. J Am Soc Nephrol 8:1568-1573.

Daly M J, et al (2001) High-resolution haplotype structure in the human genome. Nat Genet 29:229-232.

Dufton M J, Hider, R C (1977) Snake toxin secondary structure predictions: structure activity relationships. J Mol Biol 115:117-193.

Enomoto A, et al (2002) Molecular identification of a renal urate-anion exchanger that regulates blood urate levels. Nature 417: 447-452.

Fu Q, et al (2000) Molecular cloning, expression characterization, and mapping of a novel putative inhibitor of rho GTPase activity, RTKN, to D2S145-D2S286. Genomics 328-332.

Garnier J, Osguthorpe D J, Robson B (1978) Analysis of the accuracy and implications of simple methods for predicting the secondary structure of globular proteins. J Mol Biol 120:97-120.

Goldstein A M, et al (2001) A common founder for the V126D CDKN2A mutation in seven North American melanomaprone families. Br J Cancer 85:527-530.

Hedrick P W (1987) Gametic disequilibrium measures: proceed with caution. Genetics 117:331-341.

Hofmann K and Stoffel W (1993) TMbase—A database of membrane spanning proteins fragments Biol. Chem. Hoppe-Seyler 374,166.

Jaeger P (1996) Genetic versus environmental factors in renal stone disease. Curr Opin Nephrol Hypertens 5:342-346.

Jeffreys A J, Ritchie A, Neumann R (2000) High resolution analysis of haplotype diversity and meiotic crossover in the human TAP2 recombination hotspot. Hum Mol Genet 9: 725-733.

Kruglyak L, Daly M J, Lander E S (1995) Rapid multipoint linkage analysis of recessive traits in nuclear families, including homozygosity mapping. Am J Hum Genet 56:519-527.

Lahoud M H, et al (2001) Gene targeting of Desrt, a novel ARID class DNA-binding protein, causes growth retardation and abnormal development of reproductive organs. Genome Res 11(8): 1327-1334.

Laity J H, Lee B M, Wright P E (2001) Zinc finger proteins: new insight s into structural and functional diversity. Curr Opin Struct Biol 11:39-46.

Lim V I (1974) Algorithms for prediction of alpha-helical and beta-structural regions in globular proteins. J Mol Biol 88:873-894.

Lupas A, Van Dyke M, Stock J (1991) Predicting Coiled Coils from Protein Sequences Science 252:1162-1164.

Kruglyak L (1997) What is significant in whole-genome linkage disequilibrium studies? Am J Hum Genet 61: 810-812.

Malashkevich V N, et al (1996) The crystal structure of a five-stranded coiled coil in COMP: a prototype ion channel? Science 274:761-765.

Nagano K. (1977) Logical analysis of the mechanism of protein folding. IV Supersecondary structures. J. Mol. Biol. 109:235-250.

Nagase T, et al (1998) Prediction of the Coding Sequences of Unidentified Human Genes. XII. The Complete Sequences of 100 New cDNA Clones from Brain Which Code for Large Proteins in vitro. DNA Research 5: 355-364.

Ombra M N, et al (2001) Identification of a new candidate locus for uric acid nephrolithiasis. Am J Hum Genet 68: 1119-29.

Pasquier C, Hamodrakas S J (1999) An hierarchical artificial neural network system for the classification of transmembrane proteins. Protein Eng 12: 631-634.

Patil N, et al (2001) Blocks of limited haplotype diversity revealed by high-resolution scanning of human chromosome 21. Science 294:1719-1723.

Peltonen L, Palotie A, Lange K (2000) Use of population isolates for mapping complex traits. Nat Genet 1: 182-90.

Persson B, Argos P. (1996) Topology prediction of membrane proteins. Protein Sci 5: 363-371.

Ristaldi M S, et al (1989) Prenatal diagnosis of beta-thalassaemia in Mediterranean populations by dot blot analysis with DNA amplification and allele specific oligonucleotide probes. Prenat Diagn 9(9):629-38.

Rivers K, Shetty S, Menon M (2000) When and how to evaluate a patient with nephrolithiasis. Urol Clin North Am 27: 203-213.

Roch-Ramel F, Guisan B (1999) Renal transport of urate in humans. News Physiol Sci 14: 80-84.

Scheinman S J (1999) Nephrolithiasis. Semin Nephrol 19:381-388.

Serio A, Fraioli A (1999) Epidemiology of nephrolithiasis. Nephron Suppl 81:26-30.

Sobel E, Lange K (1996) Descent graphs in pedigree analysis: applications to haplotyping, location scores, and marker-sharing statistics. Am J Hum Genet 58:1323-1337.

Tabor H K, Risch N J, Myers R M (2002) Candidate-gene approaches for studying complex genetic traits: practical considerations. Nat Rev Genet 3: 391-397.

Terwilliger J D (1995) A powerful likelihood method for the analysis of linkage disequilibrium between trait loci and one or more polymorphic marker loci. Am J Hum Genet 56:777-787.

Terwilliger J D, et al (1998) Mapping in small populations with no demographic expansion. Hum Hered 48:138-154.

von Heijne G (1992) Membrane Protein Structure Prediction, Hydrophobicity Analysis and the Positive-inside Rule. J Mol Biol 225: 487-494.

Wright A F, Carothers A D, Pirastu M (1999) Population choice in mapping genes for complex diseases. Nat Genet 23:397-404.

Wu X, Lee C C, Muzny D M, Caskey C T (1989) Urate oxidase: Primary structure and evolutionary implications. Proc Natl Acad Sci USA 86, 9412-9416.

Wu X, Muzny D M, Lee C C, Caskey C T (1992) Two independent mutational events in the loss of urate oxidase. J Mol Evol 34, 78-84.

1. (canceled)

2. Nucleic acid comprising at least one fragment of the human gene ZNF365 in which said fragment encodes for a functional portion of at least one of the proteins of the ZNF365 group for use in the diagnosis or treatment of pathologies associated with kidney stones.

3. Method to detect in an individual at least one mutation in the gene encoding for one of the proteins of the human ZNF365 group located on chromosome 10 and comprising the phases:
collection of a sample containing a sufficient quantity of DNA from the aforesaid individual or reproducible in culture;
isolation of the DNA of said sample;
submission of the isolated DNA to exponential amplification using as an primer pair for amplification reaction at least two oligonucleotides that are able to amplify at least one fragment of the human gene ZNF365, in which said fragment encodes for a functional portion of at least one of the proteins of the ZNF365 group;
detection of any mutations in at least one amplified fragment compared with healthy controls.

4. Method according to claim 3 in which the DNA exponential amplification phase is performed using primer pairs for the amplification reaction that is able to amplify a part of the fragment encoding the human gene ZNF365.

5. Method according to claim 4 in which the DNA exponential amplification phase to amplify a part of the fragment encoding the human gene ZNF365 comprises the use of the following primer pairs:
Ala62Thr-F: 5′ CTC CAC TCC ACC TTT TTA AG 3′
Ala62Thr-R: 5′ GCT GAC ATT GGT ACT TAC TG 3′.

6. Method according to claim 3 in which the detection phase of any mutations in at least one amplified fragment compared with healthy controls is performed using direct sequencing.

7. Diagnostic kit for pathologies associated with kidney stones to perform the method according to claim 3 that comprises:
at least one pair of oligonucleotide primers for the exponential amplification reaction of at least one fragment of the human gene ZNF365, in which said fragment encodes for a functional portion of at least one of the proteins of the ZNF 365 group;
a control DNA from a healthy individual not affected by pathologies associated with kidney stones.

8. Kit according to claim 7 in which oligonucelotide primer pairs in the kit for performing the amplification reaction are able to amplify a part of the region encoding the gene ZNF365.

9. (canceled)

10. Protein belonging to the group of the ZNF365 proteins or a functional portion thereof for use in the diagnosis or treatment of pathologies associated with kidney stones.

11. Method according to claim 4 in which the detection phase of any mutations in at least one amplified fragment compared with healthy controls is performed using direct sequencing.

12. Method according to claim 5 in which the detection phase of any mutations in at least one amplified fragment compared with healthy controls is performed using direct sequencing.

13. Diagnostic kit for pathologies associated with kidney stones to perform the method according to claim 4 that comprises:
at least one pair of oligonucleotide primers for the exponential amplification reaction of at least one fragment of the human gene ZNF365, in which said fragment encodes for a functional portion of at least one of the proteins of the ZNF 365 group;
a control DNA from a healthy individual not affected by pathologies associated with kidney stones.

14. Diagnostic kit for pathologies associated with kidney stones to perform the method according to claim 5 that comprises:
at least one pair of oligonucleotide primers for the exponential amplification reaction of at least one fragment of the human gene ZNF365, in which said fragment encodes for a functional portion of at least one of the proteins of the ZNF 365 group;
a control DNA from a healthy individual not affected by pathologies associated with kidney stones.

15. Diagnostic kit for pathologies associated with kidney stones to perform the method according to claim 6 that comprises:
at least one pair of oligonucleotide primers for the exponential amplification reaction of at least one fragment of the human gene ZNF365, in which said fragment encodes for a functional portion of at least one of the proteins of the ZNF 365 group;
a control DNA from a healthy individual not affected by pathologies associated with kidney stones.


SEQUENCE LISTING (36 sequences)

SEQ ID NO: 1 - DNA, 20 bp, Artificial Sequence
  ZNF 365 amplification primer
ctccactccacctttttaag

SEQ ID NO: 2 - DNA, 20 bp, Artificial Sequence
  ZNF65 amplification primer
gctgacattggtacttactg

SEQ ID NO: 3 - DNA, 20 bp, Homo sapiens
gagcaagggtgttccccgcg

SEQ ID NO: 4 - DNA, 20 bp, Homo sapiens
aacaagtcgggtaagaggcg

SEQ ID NO: 5 - DNA, 20 bp, Homo sapiens
cccctcccaggacttttaga

SEQ ID NO: 6 - DNA, 20 bp, Homo sapiens
ggaaagaagagtaagtgttg

SEQ ID NO: 7 - DNA, 20 bp, Homo sapiens
tgccttgcagagaagttgtc

SEQ ID NO: 8 - DNA, 20 bp, Homo sapiens
cgggcacgtggtgagtcacc

SEQ ID NO: 9 - DNA, 20 bp, Homo sapiens
tttccttcagcttacagaca

SEQ ID NO: 10 - DNA, 20 bp, Homo sapiens
ccaaatgcctgtatgtatgt

SEQ ID NO: 11 - DNA, 20 bp, Homo sapiens
ccctctgtagaagccgaggg

SEQ ID NO: 12 - DNA, 20 bp, Homo sapiens
agctgttaagaattcctgct

SEQ ID NO: 13 - DNA, 20 bp, Homo sapiens
ttcctttcagagctggaaag

SEQ ID NO: 14 - DNA, 20 bp, Homo sapiens
ggaattggaggtaaagccac

SEQ ID NO: 15 - DNA, 20 bp, Homo sapiens
ttatgactaggagtctgcga

SEQ ID NO: 16 - DNA, 20 bp, Homo sapiens
tgagtaatttatttattaag

SEQ ID NO: 17 - DNA, 20 bp, Homo sapiens
agctttgtaagaagaggaag

SEQ ID NO: 18 - DNA, 20 bp, Homo sapiens
ctccataaccgtaagaaata

SEQ ID NO: 19 - DNA, 20 bp, Homo sapiens
ttgtttttagatttttggcc

SEQ ID NO: 20 - DNA, 20 bp, Homo sapiens
gctactgcaggtgaatactc

SEQ ID NO: 21 - DNA, 20 bp, Homo sapiens
tattttacaggaattggccc

SEQ ID NO: 22 - DNA, 20 bp, Homo sapiens
ctgtctccaggtaaattccc

SEQ ID NO: 23 - DNA, 20 bp, Homo sapiens
attattctaggtgctggaat

SEQ ID NO: 24 - DNA, 20 bp, Homo sapiens
caaaaacaaggtaagtcagt

SEQ ID NO: 25 - DNA, 20 bp, Homo sapiens
ttccacaaagtcaacacatc

SEQ ID NO: 26 - DNA, 20 bp, Homo sapiens
tcaattcaaggtcatttttg

SEQ ID NO: 27 - DNA, 20 bp, Homo sapiens
tctgtttcaggatttagcca

SEQ ID NO: 28 - DNA, 20 bp, Homo sapiens
actttgagaggtgggtgtcc

SEQ ID NO: 29 - DNA, 20 bp, Homo sapiens
atctctgcagagaaagtgtc

SEQ ID NO: 30 - DNA, 20 bp, Homo sapiens
tgtgtgattggtaagaattt

SEQ ID NO: 31 - DNA, 20 bp, Homo sapiens
gtcttcataggcaaattgac

SEQ ID NO: 32 - DNA, 20 bp, Homo sapiens
tacacagtgtatacagtttt

SEQ ID NO: 33 - protein, 407 aa, Homo sapiens
MQQKAFEESRYPWQESFENVAVCLPLRCPRCGDHTRFRSLSSLRAHLEFSHSYEERTLLTKCSLFPSLKDTDLVTSSELLKPGKLQSSGNVVKQKPSYVNLYSISHEHSKDRKPFEVVAERPVSYVQTYTAMDLHADSLDGTRSGPGLPTSDTKASFEAHVREKFNRMVEAVDRTIEKRIDKLTKELAQKTAELLEVRAAFVQLTQKKQEVQRRERALNRQVDVAVEMIAVLRQRLTESEEELLRKEEEVVTFNHFLEAAAEKEVQGKARLQDFIENLLQRVELAEKQLEYYQSQQASGFVRDLSGHVLTDISSNRKPKCLSRGHPHSVCNHPDLKSHFHPKGRNHLKKAKDDRASMQPAKAIHEQAESSRDLCRPPKKGELLGFGRKGNIRPKMAKKKPTAIVNII

SEQ ID NO: 34 - protein, 333 aa, Homo sapiens
MQQKAFEESRYPWQESFENVAVCLPLRCPRCGDHTRFRSLSSLRAHLEFSHSYEERTLLTKCSLFPSLKDTDLVTSSELLKPGKLQSSGNVVKQKPSYVNLYSISHEHSKDRKPFEVVAERPVSYVQTYTAMDLHADSLDGTRSGPGLPTSDTKASFEAHVREKFNRMVEAVDRTIEKRIDKLTKELAQKTAELLEVRAAFVQLTQKKQEVQRRERALNRQVDVAVEMIAVLRQRLTESEEELLRKEEEVVTFNHFLEAAAEKEVQGKARLQDFIENLLQRVELAEKQLEYYQSQQASGFVRDLSGHVSWKGAGEARLVCQNDLELEESAIVE

SEQ ID NO: 35 - protein, 462 aa, Homo sapiens
MQQKAFEESRYPWQESFENVAVCLPLRCPRCGDHTRFRSLSSLRAHLEFSHSYEERTLLTKCSLFPSLKDTDLVTSSELLKPGKLQSSGNVVKQKPSYVNLYSISHEHSKDRKPFEVVAERPVSYVQTYTAMDLHADSLDGTRSGPGLPTSDTKASFEAHVREKFNRMVEAVDRTIEKRIDKLTKELAQKTAELLEVRAAFVQLTQKKQEVQRRERALNRQVDVAVEMIAVLRQRLTESEEELLRKEEEVVTFNHFLEAAAEKEVQGKARLQDFIENLLQRVELAEKQLEYYQSQQASGFVRDLSGHVSWKGAGEARLVCQNDLELEIFGHINHHLSGLKDSHCLVFLQAPPVPWIILASFLWILGNPWTSSTATAGFSQIWVLFPFCGGTFHHNEKDVLGLQDFERESVSTSQSRNISLLTLGQLQNCVIGKLTIIDLLTEHLLGVRHGVICFPWGLPSSS

SEQ ID NO: 36 - protein, 216 aa, Homo sapiens
MSALGQITITVSRCWNTERNQTDKNPCLHGAYLQLRETVKNKSTHLKKPLMKQAPPWKDHLAFQPLHPAERKTQVWRWQSGNSSDLETTSSASPWPTGSNRDVVLNTLAESCCGLSELITAPPYAGVSIQGFSQIWVLFPFCGGTFHHNEKDVLGLQDFERESVSTSQSRNISLLTLGQLQNCVIGKLTIIDLLTEHLLGVRHGVICFPWGLPSSS